(19) United States
(12) Patent Application Publication
(10) Pub. No.: US 2005/0182241 A1
(43) Pub. Date: Aug. 18, 2005

(54) Universal support for nucleic acid synthesis

(75) Inventors: Nam Ngo, Campbell, CA (US); Laurent Jaquinod, Davis, CA (US); Hong Wang, San Jose, CA (US)

Correspondence Address: CT GEN, 464 S. HILLVIEW DR., MILPITAS, CA, 95051, US

(73) Assignee: CTGen, Inc., San Jose, CA (US)

(21) Appl. No.: 10/776,695

(22) Filed: Feb. 12, 2004

(51) Int. Cl. C07H 21/04; C08G 63/48; C07F 7/21

(52) U.S. Cl. 530/332; 536/23.1; 525/54.1; 556/460

(57) ABSTRACT

Polysubstituted catechol-based universal solid supports suitable for synthesizing oligonucleotides have been prepared. Following synthesis, cleavage of the oligonucleotide from the solid support and catechol-assisted elimination of the 3′-phosphate group is accomplished by treatment with standard basic media such as ammonium hydroxyde or¹ methylamine.

### REFERENCES CITED

### U.S. Patent Documents

| | | | |
|---|---|---|---|
| 4,415,732 | November, 1983 | Caruthers et al. | 536/27 |
| 4,458,066 | July, 1984 | Caruthers et al. | 536/25 |
| 4,725,677 | February, 1988 | Koster et al. | 536/27 |
| 5,047,524 | Septeber, 1991 | Andrus et al. | 536/25 |
| 6,090,934 | July, 2000 | Kumar et al. | 536/25 |
| 6,590,092 | July, 2003 | Ngo | 536/25 |

### Other References

Azhayev, A. V. Tetrahedron 1999, 55, 787-780.
A. V. Azhayev and Antopolsky, M. Tetrahedron 2001, 57, 4977-4986.
Matteuci, M. D. and Caruthers, M. H. J. Am. Chem. Soc. 1981, 3185-3191. Synthesis of deoxyoligonucleotides on a polymer support.
J. Org. Chem. 1987, 52.

### BACKGROUND OF THE INVENTION

Most current commercially available solid supports used for the solid phase synthesis of nucleic acids possess pre-attached 5′-hydroxy protected nucleosides attached to the polymer carrier via the 3′-end of its deoxyribose or ribose rings (U.S. Pat. No. 4,458,066). Organic polymers such as cross-linked polystyrene (Andrus et al) or inorganic polymers such as controlled pore glass (CPG) are preferentially used. Nucleic acids are synthesized on automated workstations by means of phosphoramidite-based coupling reagents upon selection of the appropriate nucleoside-bound solid supports. In a high throughput (HTP) format allowing up to 1564 oligonucleotides to be prepared simultaneously, sorting out nucleoside-bond solid supports is time-consuming, cumbersome and prone to deliver nucleic acids synthesized with a wrong 3′-terminal base.

Increasing use of synthetic oligonucleotides as sequencing primers, PCR primers or hybridization probes requires faster and more reliable synthetic methods. A simplification is achieved by using a universal support which are used irrespective of the first RNA or DNA nucleotide to be synthesized, and irrespective of the type of monomer reagent used during the synthesis, that is, irrespective of the type of substitution of the phosphate group in the 3′-position or in the 5′-position depending on whether the synthesis is carried out in the 5′ to 3′ or 3′ to 5′ direction. Preferably, universal solid supports allow the synthesis of nucleic acids regardless of the nature of their 3′-terminal base by reacting selectively with the 3′-end of a nucleoside, functionalized in particular with a phosphoramidite moiety. Upon oxidation, the resulting 3′-phosphate linkage must be stable under all conditions of the nucleic acid synthesis. Oligonucleotides cleaved from a universal support and deprotected are of native type that is to say that the 3′-terminal hydroxyl group does not bear any residue derived from the synthesis. In a HTP format, a universal support has further requisites: (i) the bound-oligonucleotides must be cleaved from the solid support under the standard conditions used to remove the base-labile protective groups of the exocyclic amino groups (cleavage step). Standard deprotections take place in aq. ammonium hydroxyde or aq. ammonium hydroxide-methylamine or aq. methylamine (ii) likewise, a complete elimination of the 3′-terminal phosphate group (elimination step) must take place under the timeframe and conditions used to remove the protective groups.

Previous art universal supports were prepared from aliphatic cyclic or non-cyclic adjacent cis-diols (Kumar et al.). Aliphatic diols flanked with a neighboring acetamido group to assist the 3′-phosphodiester elimination have also been used (Azhayev). Those universal supports suffer from one or more of the following shortcomings: (i) lengthy or uncomplete elimination of the 3′-universal linkers (ii) competitive side reactions such as β-elimination yielding 3′-phosphate-oligonucleotides. (iii) cleavage and elimination/deprotection carried out in two separate steps (Nucl. Ac. Res. 1996, 24, 2793). (iv) cleavage and elimination catalyzed by additional salts such as lithium chloride or lead cations (v) use of expensive nucleosidic linkers which are not incorporated into the desired nucleic acids.

We previously reported novel universal solid supports prepared from mono-substituted catechol derivatives, preferably 3-methoxycatechol (U.S. Pat. No. 6,590,092). Their drawback in HTP synthesis of nucleic acids stemmed from a lengthy catechol-asssited 3′-phosphate elimination (i.e. 3 hrs under standard conditions). The present invention describes the preparation of di-, tri- and tetra-substitutedcatechols (i.e. polysubstituted-1,2-dihydroxybenzene) based universal solid supports. Particularly, universal support prepared from 3,6-dialkoxycatechols are compatible with the high-throughput synthesis of nucleic acids as they display fast and complete 3′-phosphate elimination under standard conditions.

### BRIEF SUMMARY OF THE INVENTION

The present invention relates to the preparation of di-, tri-, and tetra-substituted catechol-based universal supports compatible to the existing methods of automated nucleic acid (DNA and RNA) synthesis employing nucleoside phosphoramidites protected with conventional or base labile groups. Those said universal supports consist of an organic or inorganic polymer (such as CPG) functionalized with a di-, tri- or tetra-substituted catechol moiety where one of the catechol group is attached to the polymer through a covalent, base-labile linkage and the other catechol group is protected by an acid labile group.

Some of the terms employed in the present description are defined subsequently, after which the invention is explained in detail. The term “nucleic acid” refers to ribonucleic acids or deoxyribonucleic acid or oligonucleotides in which modifications can take place at the level of the base (generating modified products such as inosine, methyl-5-deoxycytidine, deoxyuridine, dimethylamino-5-deoxyuridine, diamino-2,6-purine or bromo-5-deoxyuridinthe), the ribose rings or the internucleotide phosphate bonds in a chemically known manner. They may in particular be oligonucleotide of alpha- or beta-anomers, -oligonucleotides of the phosphorothioate or alkyl phosphonate or boronate type. Depending on whether the nucleic acid is DNA or RNA, the nitrogen base is selected from adenine, guanine, uracil, cytosine and thymine. Protective groups are those conventionally employed in the chemical synthesis of nucleosides, nucleotides and oligonucleotides (see, for example: Protocols for Oligonucleotides and Analogs, Synthesis and Properties, edited by Sudhir Agrawal, Humana Press, Totowa, N.J.).

### BRIEF DESCRIPTION OF THE SEVERAL VIEWS OF THE DRAWING

FIG. 1 describes the catechol-assisted 3′-phosphate elimination yielding 3′-hydroxyoligonucleotides.

FIG. 2 describes the preparation of 3,6-dimethoxycatechol based universal support 4.

### DETAILED DESCRIPTION OF THE INVENTION

The present invention describes the preparation of novel universal supports comprising a polysubstituted (di-, tri- or tetra-) catechol moiety where one of the hydroxyl groups is attached to a polymeric support through a base-labile, covalent linkage. The said substrates, useful for the synthesis of nucleic acids, have the formulae shown structure 1 wherein:
W is a polymer support that may be selected from organic polymers such as cross-linked polystyrene or inorganic polymers such as silica gel (porous or non-porous) or controlled pore glass (CPG).
L is a base-labile, (C, N, O) containing linker arm covalently attaching the polymer carrier to the polysubstituted catechol moiety.
R1 and R4: H, alkoxy, N(alkyl)₂, alkyl, halogen (F, Cl, Br);
R2 and R3: H, alkoxy, alkyl, halogen (F, Cl, Br);
R5: H, Si(alkyl)₃.

As used herein, the term alkyl refers to straight-chained such as propyl, branched or cyclic alkyls from 1 to 18 carbons and the term alkoxy refers to methoxy, ethoxy, propoxy or the like.

In a particularly preferred embodiment, the inventive substrates are 3,6-dialkoxycatechols-bound CPG which structures are shown structure 2, wherein:
W=CPG;
n: 1 to 18;
R1 and R4=alkyl;
R2 and R3: H, alkyl;
R5=Si(alkyl)₃
R6=H, alkyl.

The preparation of 3,6-dialkoxycatechol-based universal supports comprises the following steps:

(i) Preparing a low loading capacity [(alkylamino)alkyl]CPG by reacting CPG with [(alkylamino)alkyl]trialkoxysilane. Preferentially, a low loading capacity (10-40 μmol/g) aminopropylCPG 3 is prepared by reacting CPG with aminopropyltriethoxysilane in dichloromethane. CPG is available from numerous commercial sources (for instance from CPG Inc, Silicycle, Fuji Silysia Chemical LTD, Prime Synthesis Inc . . . ). The beads are in the size range of 75-200 microns and contain pores having substantially similar diameter in the range between 100-4000 angstroms. Preferably, the pore diameters are about 1000 angstroms.

(ii) Reacting carbonyldiimidazole (CDI) with 3,6-dialkoxycatechol.

(iii) Reacting aminoalkylCPG with a 3,6-dialkoxycatecholcarbonate prepared step (ii). To ensure complete reaction of the amino groups, the phenoxycarbonylation reaction is carried out in the presence of an excess of carbonate. Completeness of the reaction is controlled with the Kaiser or ninhydrin tests.

(iv) Protecting and capping CPG catechol and silanol groups simultaneously with excess trialkylsilylimidazole. Preferentially, trimethylsilylimidazole is used.

The inventive 3,6-dialkoxycatechol-based universal supports are employed to synthesize nucleic acids, in particular via the automated synthesis, using conventional cyanoethylphosphoramidite chemistry (U.S. Pat. Nos. 4,725,677; BioTechniques Vol. 22, No. 4, 752-756, 1997) and conventional or labile nucleotide protecting groups. Prior to starting a synthesis, the trimethylsilyl groups protecting the catechol groups are cleaved by washing catechol-bound CPG with a solution of dichloroacetic acid or trichloroacetic acid in dichloromethane. On an automated workstation, the trimethylsilyl groups are cleaved under the standard conditions used, preferentially 3% dichloroacetic acid in dichloromethane. The first DMT-protected nucleoside phosphoramidite is then reacted to the catechol-bound support under the standard conditions familiar to those skilled in the art. The resulting phosphodiester linkage is oxidized to the corresponding phosphate with 0.1M iodine in H₂O/pyridine/THF. Chain elongation occurs by sequential reaction of 5′-protected nucleoside phosphoramidites with the 5′-hydroxyl-end of the oligonucleotide bound polymer. One synthetic cycle involves the deprotection of the 5′-hydroxyl group of the nucleotide bound polymer, its condensation with a 3′-phosphoramidite nucleotide, and finally oxidation of the resulting phosphodiester internucleotide linkage. Additional experimental details can be downloaded from www.ctgen.com.

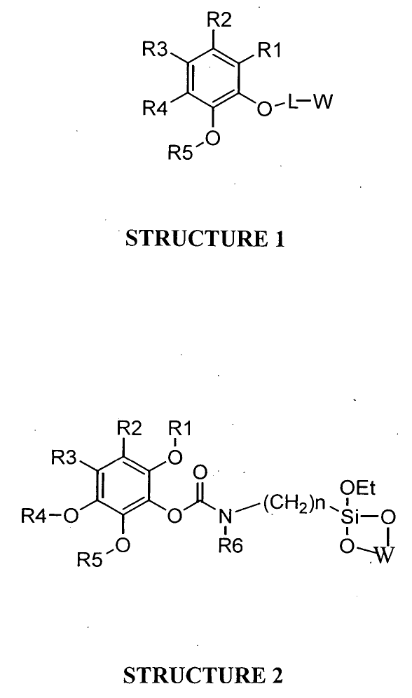

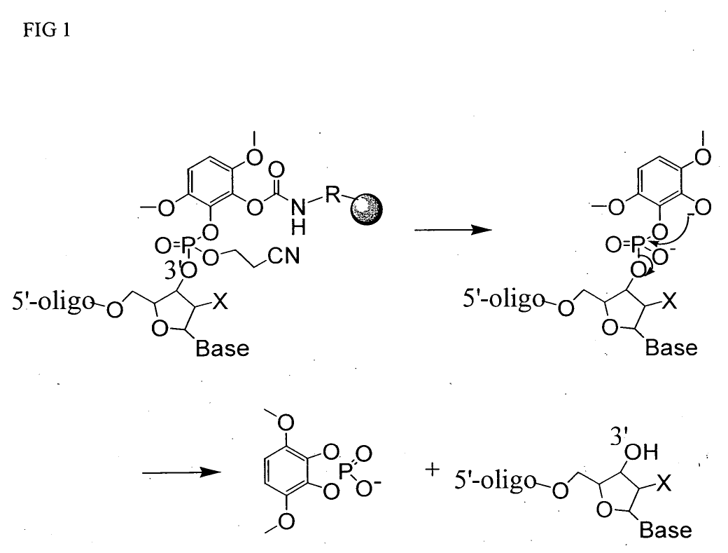

Upon completion of the synthesis, the nucleic acids (both DMT-on full length and failure sequences) are cleaved from the solid support while the concomitant elimination of the catechol assisted 3′-phosphate group and the removal of the protecting groups from the bases take place simultaneously. Hydrolitic cleavages commonly used are those selected from 33% ammonium hydroxide, 40% aq. methylamine or ammonia-methylamine (1:1/v:v) which at 80° C. ensure a complete 3′-phosphate elimination in 1 hr. 40 nm and 30 min, respectively. 3′-Catechol assisted phosphate elimination via the formation of a cyclic catechol phosphate is shown FIG. 1.

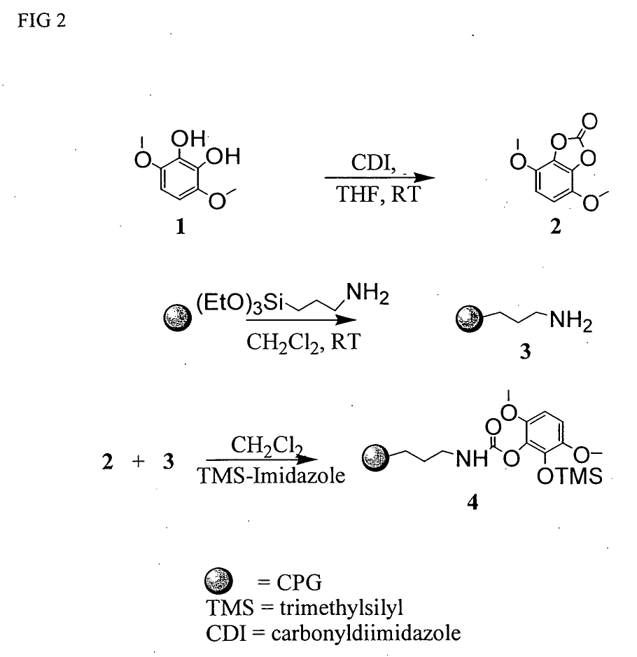

The following examples serve to illustrate the present invention and provides additional details concerning the preparation (as shown FIG. 2) of a 3,6-dimethoxycatechol-based universal support 4. It is not intended to be exhaustive or to limit the invention to the precise form or reaction schemes disclosed. Obviously, many modifications and variations are possible in light of the above teaching.

### EXAMPLE 1

Dimethoxycatechol 1 was prepared in four steps from 2,5-dimethoxybenzaldehyde according to the literature. AminopropylCPG 3 was prepared by reacting CPG (75/200, 1000 angstroms pore size) with aminopropyltriethoxysilane in dichloromethane.

In a 200 mL round bottomed flask under argon, dimethoxycatechol 1 (305 mg, 1.8 mmol) was dissolved in dried THF (20 mL). Carbonyldiimidazole (1.02 equiv) was added at once. The reaction mixture was stirred for 30 min at room temperature, diluted with dichloromethane (20 mL) and added under argon to a suspension of aminopropylCPC 3 (10 g, 30 μmol/g) in dichloromethane. The flask was shaken at room temperature overnight. Additional carbonate 2 is added if a ninhydrin test detecting free amino groups is positive. Trimethylsilylimidazole (0.7 mL) is added and the flask is shaked for another two hours. Methanol (10 mL) is added and the flask is shaked for another 10 min. Dimethoxycatechol-CPG 4 is filtered, washed with methanol (2×) and dichloromethane (2×) and dried under vacuum.

### EXAMPLE 2

### Deprotection with Ammonium Hydroxide

A support bound 25-mer DMT-on oligonucleotide was synthesized using universal solid support 4 (200 nmol, 10 mg), phosphoramidite chemistry and conventional protective groups by standard techniques known to those skilled in the art. To the 25-mer bound CPG in a 2 mL vial was added 1 mL of 30% ammonium hydroxide. The vial was sealed and heated at 80° C. for 60 mn. The supernatant solution was separated from the CPG support, which was washed with concentrated aq. ammonium hydroxide and discarded. The combined ammonium hydroxyde solutions were combined and analyzed by HPLC. The fully deprotected DMT-on 25-mer oligonucleotide was found to be identical to samples prepared from conventional commercial supports. HPLC analyses were carried out using a OPH® RP-L21 column (Organicphase Inc., 3.0×75 mm, particle size 5 μm). Sample volume was 10 μL using a flow rate of 0.75 mL/min. The column was equilibrated in buffer A (0.1M TEAA, pH 7.0) and eluted in a gradient of buffer B (H₂O/acetonitrile, 1:3, v/v).

1. A universal solid support comprising a polysubstituted-1,2-dihydroxybenzene where one of the hydroxyl groups is covalently attached to the polymer carrier through a base labile, covalent linkage and the other hydroxyl group is protected by an acid labile group.

2. A coumpound of claim 1, wherein said polysubstituted-1,2-dihydroxybenzene is a di-, tri-, or tetra-substituted catechol defined by the formula 3-(R1),4-(R2),5-(R3),6-(R4)-1,2-dihydroxybenzene.

3. A compound of claim 2, wherein R1 and R4 are selected from the group consisting of hydrogen, lower alkyl, and alkoxy and wherein R2 and R3 are selected from the group consisting of hydrogen, lower alkyl, alkoxy and halogen.

4. A compound of claim 3, wherein R1 and R4 are selected from the group consisting of methoxy, ethoxy, propoxy, butoxy and biphenoxy and wherein R2 and R3 are selected from the group consisting of hydrogen, methyl, ethyl, propyl, methoxy and ethoxy.

5. A compound of claim 4, wherein R1 and R4 are selected from the group consisting of methoxy and ethoxy and wherein R2 and R3 are selected from the group consisting of hydrogen and methyl.

6. A compound of claim 1, wherein the said acid labile group is selected from the group consisting of -silyl(alkyl)_{3.}

7. A compound of claim 6, wherein the said alkyl is methyl.

8. A compound of claim 1, wherein the said polymer carrier is controlled porous glass (CPG).
